(19) United States
(12) Patent Application Publication
(10) Pub. No.: US 2025/0224387 A1
(43) Pub. Date: Jul. 10, 2025

(54) AMMONIA GAS SENSOR BASED ON SMALL-PERIOD LONGPERIOD FIBER GRATING

(71) Applicant: HUAZHONG UNIVERSITY OF SCIENCE AND TECHNOLOGY, Wuhan (CN)

(72) Inventors: Xuewen SHU, Wuhan (CN); Hanyuan LIU, Wuhan (CN); Lin ZHANG, Birmingham (GB)

(73) Assignee: HUAZHONG UNIVERSITY OF SCIENCE AND TECHNOLOGY, Wuhan (CN)

(21) Appl. No.: 18/970,997

(22) Filed: Dec. 6, 2024

Related U.S. Application Data
(63) Continuation of application No. PCT/CN2024/074396, filed on Jan. 29, 2024.

(30) Foreign Application Priority Data
Jan. 8, 2024 (CN) 202410029277.0

(51) Int. Cl. G01N 33/00; G01K 11/3206; G01N 21/41

(52) U.S. Cl. CPC G01N 33/0054; G01K 11/3206; G01N 21/412

(57) ABSTRACT

An ammonia gas sensor based on a small-period long-period fiber grating is provided. The ammonia gas sensor based on a small-period long-period fiber grating includes a small-period long-period fiber grating and a gas sensitive layer coated on an external surface of a single-mode fiber, the small-period long-period fiber grating is a fiber grating structure inscribed at the interior of the single-mode fiber, the cladding surface of small-period long-period fiber grating is a refractive index sensitive area, the gas sensitive layer adopts a high molecular polymer, and a refractive index of the high molecular polymer changes with the change of ammonia concentration for ammonia detection. The ammonia gas sensor of the present disclosure has the advantages of compact structure, high sensitivity, good stability, short response time, low production difficulty, and strong anti-interference ability, and the ammonia gas sensor may monitor the ambient temperature simultaneously.

### CROSS REFERENCE TO THE RELATED APPLICATIONS

This application is a continuation application of International Application No. PCT/CN2024/074396, filed on Jan. 29, 2024, which is based upon and claims priority to Chinese Patent Application No. 202410029277.0, filed on Jan. 8, 2024, the entire contents of which are incorporated herein by reference.

### TECHNICAL FIELD

The present disclosure relates to the technical field of optical fiber sensing, and in particular to an ammonia gas sensor based on a small-period long-period fiber grating.

### BACKGROUND

Ammonia gas has been widely used in many industries such as chemistry, agriculture, food, electronic, and so on. However, due to its harm to human beings, various sensors have been developed to detect the concentration of Ammonia gas by researchers, to achieve early warning. Normal ammonia gas sensors can be mainly divided into electrochemical, semiconductor, solid electrolyte, spectrum absorption, contact combustion and optical fiber contact. Semiconductor gas sensors have the disadvantages of high operating temperature, easy to be affected by electromagnetic interference, and relatively low detection accuracy, which is difficult to detect low concentration gas of ppb level; and spectral absorption gas sensors can overcome the problems of electrical gas sensing, but its detection limit and detection sensitivity are limited by the length of the gas chamber optical path, and the longer gas chamber optical path limits the miniaturization and integration of the sensor.

With the advantages of compact structure, high sensitivity, anti-electromagnetic interference, anti-corrosion and remote monitoring, the optical fiber contact ammonia gas sensor has been deeply studied by researchers. At present, the fiber structures that have been reported include a D-shaped fiber, a micro-nano fiber, a tapered fiber, a long-period fiber grating and a tilted fiber Bragg grating. However, the D-shaped fiber, the micro-nano fiber, the tapered fiber all have the disadvantage of fragile structure, and the long-period fiber grating and the tilted fiber Bragg grating are easily affected by the temperature variations and cannot monitor temperature at the same time, which may lead to errors in measurement.

Therefore, it is urgent for technicians in this field to provide an ammonia sensor that can simultaneously monitor ambient temperature and ammonia concentration based on a small-period long-period fiber grating.

### SUMMARY

A purpose of the present disclosure is to provide an ammonia gas sensor based on a small-period long-period fiber grating, which is used to solve the problem that traditional fiber optic contact ammonia gas sensors are easily affected by ambient temperature crosstalk and cannot simultaneously measure environmental temperature and ammonia concentration

In order to solve the above problem the present disclosure adopts the following technical solutions.

The present disclosure provides an ammonia gas sensor based on a small-period long-period fiber grating, including a small-period long-period fiber grating and a gas sensitive layer coated on an external surface of a single-mode fiber, a position of the small-period long-period fiber grating corresponds to a position of the gas sensitive layer, the small-period long-period fiber grating is a fiber grating structure inscribed at the interior of the single-mode fiber, the cladding surface of small-period long-period fiber grating is a refractive index sensitive area, the gas sensitive layer adopts a high molecular polymer, and a refractive index of the high molecular polymer changes with the change of ammonia concentration for ammonia detection.

Preferably, the high molecular polymer is poly(diallyldimethylammonium chloride) and poly(acrylic acid).

Preferably, the small-period long-period fiber grating is obtained by inscribing at the interior of the single-mode fiber without the coating layer through femtosecond laser adopting a line-by-line method, a transmission spectrum of the small-period long-period fiber grating contains high-order cladding modes, and a reflection spectrum of the small-period long-period fiber grating contains a series of high-order Bragg reflection peaks.

A preparation method of the gas sensitive layer of the ammonia gas sensor based on the small-period long-period fiber grating includes the following steps:
step 1: soaking a small-period long-period fiber grating using an alkaline solution to ionize the surface of the small-period long-period fiber grating;
step 2: soaking a product obtained in step 1 by adopting a positively charged poly(diallyldimethylammonium chloride) solution, to facilitate better binding of poly(acrylic acid) to the surface of a single-mode fiber;
step 3: soaking a product obtained in step 2 by adopting deionized water, and drying the product with nitrogen, to remove residual molecular groups;
step 4: soaking a product obtained in step 3 by adopting a negatively charged poly(acrylic acid) solution, to combine a gas sensitive layer with ammonia gas;
step 5: soaking a product obtained in step 4 by adopting deionized water, and drying the product with nitrogen, to remove residual molecular groups; and
step 6: repeating steps 2-5, to make a thickness of the gas sensitive layer reach 100 nm-500 nm.

An ammonia concentration test system of the ammonia gas sensor based on the small-period long-period fiber grating includes a broadband light source, an optical circulator, a gas chamber, an ammonia gas sensor based on a small-period long-period fiber and a spectrometer; the broadband light source, the optical circulator, the ammonia gas sensor based on the small-period long-period fiber and the spectrometer are arranged and connected in sequence, the ammonia gas sensor based on the small-period long-period fiber is placed in the gas chamber, emitted light of the broadband light source is transmitted to the ammonia gas sensor based on the small-period long-period fiber through the optical circulator, an refractive index of the gas sensitive layer of the ammonia gas sensor based on the small-period long-period fiber responds to the change of ammonia concentration, and the ammonia concentration information is displayed by the spectrometer.

An ambient temperature test system the ammonia gas sensor based on the small-period long-period fiber grating includes a broadband light source, an optical circulator, an ammonia gas sensor based on a small-period long-period fiber, a spectrometer and a tube furnace, the broadband light source, the ammonia gas sensor based on the small-period long-period fiber, the spectrometer, and the optical circulator are arranged and connected according to positions, the ammonia gas sensor based on the small-period long-period fiber is placed in the tube furnace, emitted light of the broadband light source is transmitted to the ammonia gas sensor based on the small-period long-period fiber through the optical circulator, a wavelength of the high-order Bragg resonance peak in the reflection spectrum of the ammonia gas sensor based on the small-period long-period fiber changes with temperature for temperature detection, and the temperature information is displayed by the spectrometer.

Compared with the prior art, the present disclosure has beneficial technical effects:
(1) The device length of the ammonia gas sensor based on the small-period long-period fiber of the present disclosure is shorter than that of the traditional long period fiber grating, and it is easier to miniaturize and integrate the sensing system.
(2) Compared with the complex, time-consuming and high-cost thin film deposition techniques such as magnetron sputtering, physical vapor deposition and chemical vapor deposition, the layer-by-layer electrostatic self-assembly film-forming method of the present disclosure is simple and efficient, and the film is uniform and the thickness may be adjusted.
(3) The precision machining technology of femtosecond laser of the present disclosure can flexibly process fiber gratings with different parameters, with short processing time and high processing repeatability.
(4) The present disclosure may not only realize the detection of ammonia concentration, but also monitor the ambient temperature change at the same time.

In summary, the ammonia gas sensor based on the small-period long-period fiber grating has the advantages of compact structure, high sensitivity, good stability, short response time, low production difficulty, low cost and strong anti-interference ability, and the ammonia gas sensor based on the small-period long-period fiber grating may monitor the ambient temperature simultaneously and overcome the influence of temperature fluctuation on ammonia gas detection.

### BRIEF DESCRIPTION OF THE DRAWINGS

The following is a further explanation of the present disclosure in combination with drawings.

FIG. 1 is an assembly schematic diagram of an ammonia gas sensor based on a small-period long-period fiber grating of the present disclosure;

FIG. 2 is a schematic diagram of an ammonia concentration test system of the present disclosure;

FIG. 3 is a schematic diagram of an ambient temperature test system of the present disclosure;

FIG. 4 shows a transmission spectrum and a reflection spectrum of a small-period long-period fiber grating of the present disclosure;

FIG. 5 shows transmission spectrums of an ammonia gas sensor made by the present disclosure at different concentrations of ammonia;

FIG. 6 is a change of a wavelength of cladding mode resonances of a small-period long-period fiber grating with a concentration of ammonia;

FIG. 7 shows reflection spectrums of an ammonia gas sensor made by the present disclosure at different temperatures; and

FIG. 8 is a change of a wavelength of Bragg resonance peaks of a small-period long-period fiber grating with a temperature.

List of reference characters: 11. single-mode fiber; 12. gas sensitive layer; 13. small-period long-period fiber grating; 21. broadband light source; 22. optical circulator; 23. gas chamber; 24. ammonia gas sensor based on the small-period long-period fiber; 25. spectrometer; and 31. tube furnace.

### DETAILED DESCRIPTION OF THE EMBODIMENTS

In order to make the technical problems, technical solutions and beneficial effects of the present disclosure more clear and clear, in the following, the present disclosure will be further detailed in combination with the drawings and embodiments. It should be understood that the specific embodiments described here are only used to explain the present disclosure and are not used to limit the present disclosure.

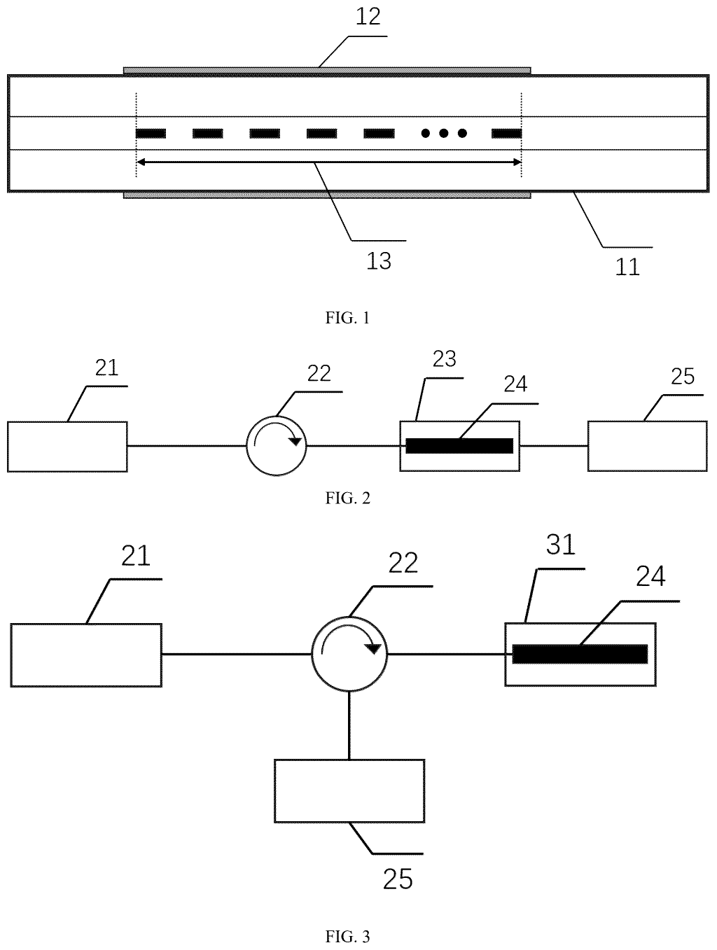

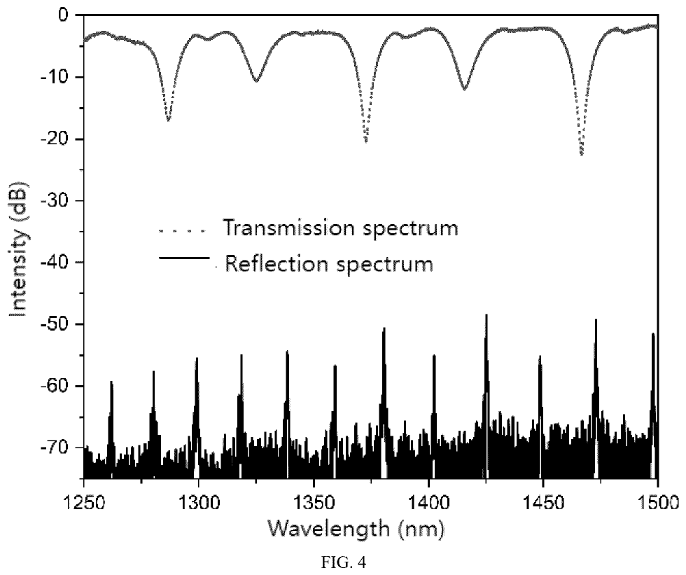

As shown in FIGS. 1-8, an ammonia gas sensor based on a small-period long-period fiber grating, including a small-period long-period fiber grating 13 and a gas sensitive layer 12 coated on an external surface of a single-mode fiber 11, a position of the small-period long-period fiber grating 13 corresponds to a position of the gas sensitive layer 12, the small-period long-period fiber grating 13 is a fiber grating structure inscribed at the interior of the single-mode fiber 11, the cladding surface of the small-period long-period fiber grating 13 is a refractive index sensitive area, the gas sensitive layer 12 adopts high molecular polymer, and a refractive index of the high molecular polymer changes with the change of ammonia concentration for ammonia detection.

Specifically, a working principle of the present disclosure is: the poly(diallyldimethylammonium chloride)/poly(acrylic acid) film is deposited on the surface of small-period long-period fiber grating via a layer-by-layer (LBL) electrostatic self-assembly method, the carboxyl functional group of poly(acrylic acid) may react with ammonia, resulting in a change in the refractive index of the coating layer, and this change may be detected by the cladding modes of the small-period long-period fiber grating 13. Because the period of the small period fiber grating 13 inscribed by femtosecond laser is only 30 μm, which is far smaller than the traditional long-period fiber grating period of hundreds of microns, therefore, the small-period long-period fiber grating 13 not only has a series of high-order transmission peaks in the transmission spectrum, but also has a series of Bragg reflection peaks in the reflection spectrum. The concentration of ammonia is detected by the wavelength shifting of the transmission peaks, and the ambient temperature is detected by the wavelength shifting of the Bragg reflection peaks. Because the Bragg reflection peaks are not sensitive to ammonia, the influence of temperature on the wavelength shifting of the transmission peaks may be compensated by the relationship between the wavelength of the Bragg reflection peaks and temperature, therefore the concentration of ammonia and the ambient temperature may be measured simultaneously.

Wherein one specific embodiment is: the small-period long-period fiber grating 13 is a fiber grating structure with a period of 30 μm, a duty cycle of 50%, and a length of 3 mm written by a femtosecond laser in a single-mode fiber 11, and the gas sensitive layer 12 is a high molecular polymer poly(diallyldimethylammonium chloride) and poly(acrylic acid with a thickness of 120 nm.
the high molecular polymer is poly(diallyldimethylammonium chloride) (PDDA) and poly(acrylic acid) (PAA).

The small-period long-period fiber grating 13 is obtained by inscribing at the interior of the single-mode fiber 11 without the coating layer through femtosecond laser adopting a line-by-line method, a transmission spectrum of the small-period long-period fiber grating 13 contains high-order cladding mode, and a reflection spectrum of the small-period long-period fiber grating 13 contains a series of high-order Bragg reflection peaks.

A preparation method of the gas sensitive layer of the ammonia gas sensor based on the small-period long-period fiber grating includes the following steps:
step 1: soaking a small-period long-period fiber grating using an alkaline solution to ionize the surface of the small-period long-period fiber grating;
step 2: soaking a product obtained in step 1 by adopting a positively charged poly(diallyldimethylammonium chloride) solution, to facilitate better binding of poly(acrylic acid) to the surface of a single-mode fiber;
step 3: soaking a product obtained in step 2 by adopting deionized water, and drying the product with nitrogen, to remove residual molecular groups;
step 4: soaking a product obtained in step 3 by adopting a negatively charged poly(acrylic acid) solution, to combine a gas sensitive layer with ammonia gas;
step 5: soaking a product obtained in step 4 by adopting deionized water, and drying the product with nitrogen, to remove residual molecular groups; and
step 6: repeating steps 2-5, to make a thickness of the gas sensitive layer reach 100 nm-500 nm.

Wherein, in a specific embodiment, a preparation steps of the gas sensitive layer are:
step 1: soaking a small-period long-period fiber grating using a potassium hydroxide solution for 30 min, to ionize the surface of the small-period long-period fiber grating;
step 2: soaking a product obtained in step 1 by adopting a positively charged poly(diallyldimethylammonium chloride) solution (0.5 wt. %, and pH=3.1), to facilitate better binding of poly(acrylic acid) to the surface of a single-mode fiber; and specifically, wt. % is a weight content percentage;
step 3: soaking a product obtained in step 2 by adopting deionized water, and drying the product with nitrogen, to remove residual molecular groups;
step 4: soaking a product obtained in step 3 by adopting a negatively charged poly(acrylic acid) solution (0.05 wt. %, and pH=4.2), to combine a gas sensitive layer with ammonia gas;
step 5: soaking a product obtained in step 4 by adopting deionized water, and drying the product with nitrogen, to remove residual molecular groups; and
step 6: repeating steps 2-5, to make a thickness of the gas sensitive layer reach 100 nm-500 nm.

As shown in FIG. 2, an ammonia concentration test system of the ammonia gas sensor based on the small-period long-period fiber grating includes a broadband light source 21, an optical circulator 22, a gas chamber 23, an ammonia gas sensor based on a small-period long-period fiber 24 and a spectrometer 25; the broadband light source 21, the optical circulator 22, the ammonia gas sensor based on the small-period long-period fiber 24 and the spectrometer 25 are arranged and connected in sequence, the ammonia gas sensor based on the small-period long-period fiber 24 is placed in the gas chamber 23, emitted light of the broadband light source 21 is transmitted to the ammonia gas sensor based on the small-period long-period fiber 24 through the optical circulator 22, an refractive index of the gas sensitive layer 12 of the ammonia gas sensor based on the small-period long-period fiber 24 responds to the change of ammonia concentration, and the ammonia concentration information is displayed by the spectrometer.

A working principle of the ammonia concentration test system is: the light emitted from the broadband light source 21 passes through the circulator 22 to the ammonia gas sensor based on the small-period long-period fiber 24; a change of ammonia concentration in the gas chamber 23 changes the refractive index of the gas sensitive layer 12, and the refractive index change is reflected by the cladding modes in the transmission spectrum of the ammonia gas sensor based on the small-period long-period fiber 24; the transmitted light containing the ammonia concentration information is received by the spectrometer 25, and the ammonia concentration information is obtained by the change of a resonant wavelength of cladding modes in the transmission spectrum of the ammonia gas sensor based on the small-period long-period fiber 24.

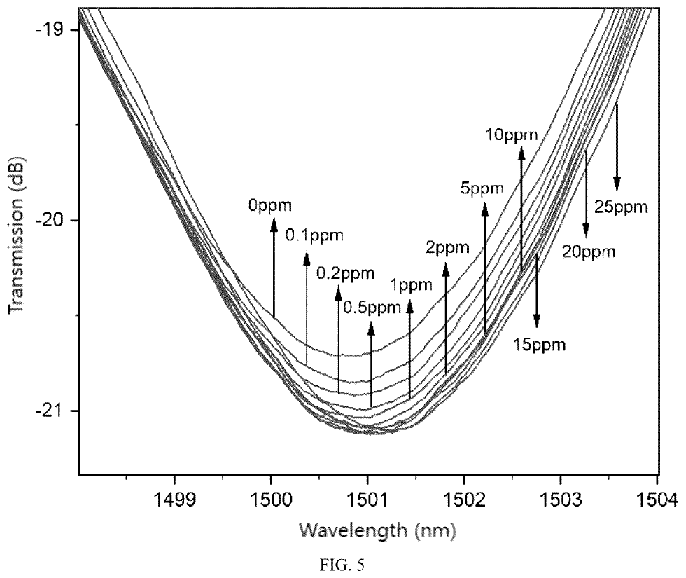

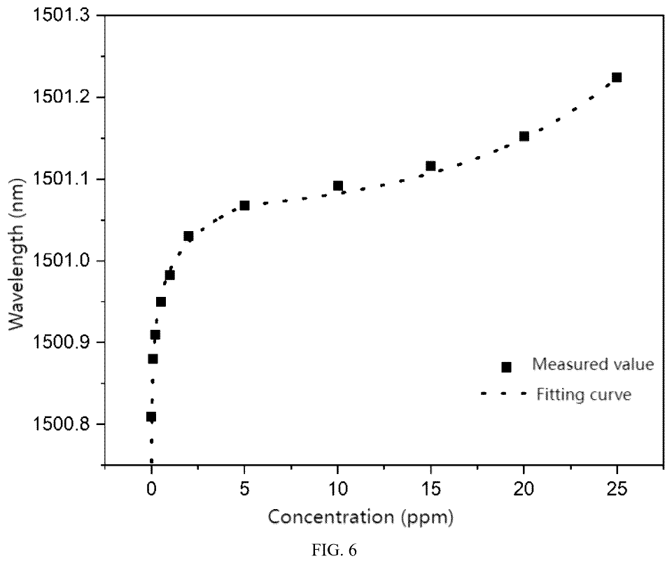

FIG. 5 shows transmission spectrums of an ammonia gas sensor based on the small-period long-period fiber 24 at different concentrations of ammonia, a measurement range is 0.1 ppm-25 ppm, and when the ammonia concentration is 0.1 ppm, the resonant peak wavelength of the cladding modes has a response; FIG. 6 is a change of a cladding mode resonance wavelength of a small-period long-period fiber grating 24 with a concentration of ammonia, the figure shows a logarithmic change at lower concentrations, while the figure shows an exponential change at higher concentrations. It can be seen from FIG. 6 that the curve fitting degree is better, which is in line with the trend of gas sensing based on refractive index change; and FIG. 6 shows that a curve fitting degree is better, which conforms to the trend of gas sensing based on refractive index changes.

As shown in FIG. 3, an ambient temperature test system the ammonia gas sensor based on the small-period long-period fiber grating includes a broadband light source 21, an optical circulator 22, an ammonia gas sensor based on a small-period long-period fiber 24, a spectrometer 25 and a tube furnace 31, the broadband light source 21, the ammonia gas sensor based on the small-period long-period fiber 24, the spectrometer 25, and the optical circulator 22 are arranged and connected according to positions, the ammonia gas sensor based on the small-period long-period fiber 24 is placed in the tube furnace 31, emitted light of the broadband light source 21 is transmitted to the ammonia gas sensor based on the small-period long-period fiber 24 through the optical circulator 22, a wavelength of the high-order Bragg resonance peak in the reflection spectrum of the ammonia gas sensor based on the small-period long-period fiber 24 changes with temperature for temperature detection, and the temperature information is displayed by the spectrometer 25.

A working principle of the ambient temperature test system is: the light emitted from the broadband light source 21 passes through the optical circulator 22 to the ammonia gas sensor based on the small-period long-period fiber 24, by adjusting a temperature in the tube furnace 31, a wavelength of the high-order Bragg resonance peaks in the reflection spectrum of the ammonia gas sensor based on the small-period long-period fiber 24 may change with the temperature; the reflected light containing temperature information is received by the spectrometer 25 through the optical circulator 22, and the environmental temperature information is obtained by the change of the wavelength of the high-order Bragg resonance peaks in the reflection spectrum of the ammonia gas sensor based on the small-period long-period fiber 24 on the spectrometer 25.

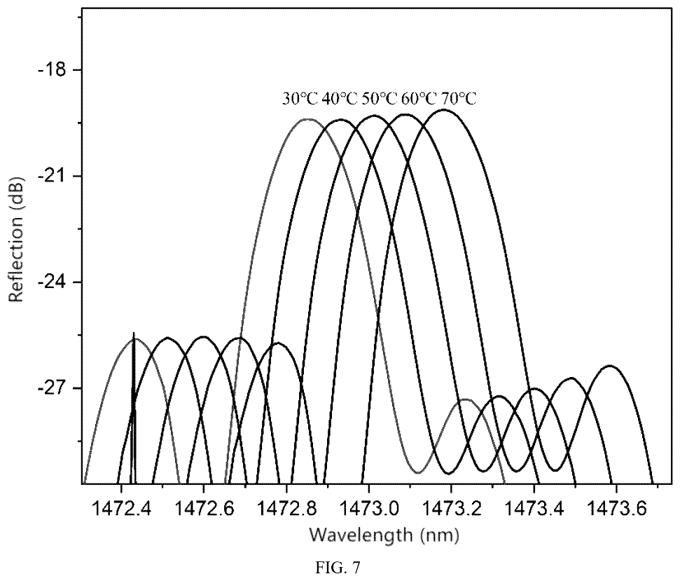

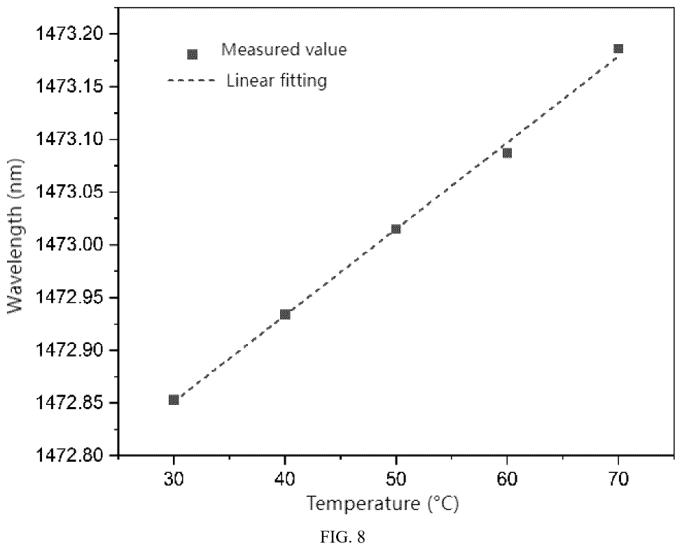

FIG. 7 shows reflection spectrums of an ammonia gas sensor made by the present disclosure at different temperatures, a measuring range is 30° C.-70° C.; FIG. 7 shows that with the increase of temperature, the wavelength of Bragg reflection peaks gradually redshifts (moving to long wavelengths); on the contrary, as the temperature decreases, the wavelength of the Bragg reflection peaks gradually blue shifts (moving to shorter wavelengths); FIG. 8 is a change of a wavelength of a Bragg resonance peak of a small-period long-period fiber grating 24 with a temperature, the R² of a linear fitting is 0.999, which explains shows that the wavelength of high-order Bragg resonance peaks has a better linear relationship with the change of ambient temperature, a slope of the linear fitting indicates that a temperature sensitivity of the sensor is 0.0087 nm/° C., therefore, the actual ambient temperature value can be determined according to a measured wavelength value of the Bragg reflection peak in practical applications.

It is important to note that in this article, relational terms such as first and second are used only to distinguish an entity or operation from another entity or operation, and do not necessarily require or imply the existence of any such actual relationship or order between those entities or operations. Moreover, the term “include”, “comprise” or any other variation thereof is intended to cover non-exclusive inclusion, thereby making a process, method, article or device that includes a range of elements not only include those elements, but also include other elements that are not clearly listed, or include the inherent elements of the process, method, object or equipment.

The above embodiments merely describe the preferred method of the present disclosure, and do not limit the scope of the present disclosure, on the premise of not departing from the design spirit of the present disclosure, all kinds of deformations and improvements made by ordinary technicians in this field to the technical scheme of the disclosure should be within the scope of protection determined by the claim of the present disclosure.

What is claimed is:

1. An ammonia gas sensor based on a small-period long-period fiber grating, comprising the small-period long-period fiber grating and a gas sensitive layer coated on an external surface of a single-mode fiber, wherein a position of the small-period long-period fiber grating corresponds to a position of the gas sensitive layer, the small-period long-period fiber grating is a fiber grating structure inscribed at an interior of the single-mode fiber, a cladding surface of the small-period long-period fiber grating is a refractive index sensitive area, the gas sensitive layer adopts a high molecular polymer, and a refractive index of the high molecular polymer changes with a change of an ammonia concentration for an ammonia detection.

2. The ammonia gas sensor according to claim 1, wherein the high molecular polymer of the gas sensitive layer is poly(diallyldimethylammonium chloride) and poly(acrylic acid).

3. The ammonia gas sensor according to claim 1, wherein the small-period long-period fiber grating is obtained by inscribing at the interior of the single-mode fiber without a coating layer through a femtosecond laser adopting a line-by-line method, a transmission spectrum of the small-period long-period fiber grating contains high-order cladding modes, and a reflection spectrum of the small-period long-period fiber grating contains a series of high-order Bragg reflection peaks.

4. A preparation method of the gas sensitive layer of the ammonia gas sensor according to claim 1, comprising the following steps:
step 1: soaking the small-period long-period fiber grating using an alkaline solution to ionize a surface of the small-period long-period fiber grating;
step 2: soaking a product obtained in the step 1 by adopting a positively charged poly(diallyldimethylammonium chloride) solution, to facilitate a better binding of poly(acrylic acid) to a surface of the single-mode fiber;
step 3: soaking a product obtained in the step 2 by adopting deionized water, and drying a soaked product obtained in the step 3 with nitrogen, to remove first residual molecular groups;
step 4: soaking a product obtained in the step 3 by adopting a negatively charged poly(acrylic acid) solution, to combine the gas sensitive layer with ammonia gas;
step 5: soaking a product obtained in the step 4 by adopting the deionized water, and drying a soaked product obtained in the step 5 with the nitrogen, to remove second residual molecular groups; and
step 6: repeating the steps 2-5, to make a thickness of the gas sensitive layer reach 100 nm-500 nm.

5. An ammonia concentration test system of the ammonia gas sensor according to claim 1, comprising a broadband light source, an optical circulator, a gas chamber, the ammonia gas sensor based on the small-period long-period fiber grating, and a spectrometer; wherein the broadband light source, the optical circulator, the ammonia gas sensor based on the small-period long-period fiber grating, and the spectrometer are arranged and connected in sequence, the ammonia gas sensor based on the small-period long-period fiber grating is placed in the gas chamber, an emitted light of the broadband light source is transmitted to the ammonia gas sensor based on the small-period long-period fiber grating through the optical circulator, an refractive index of the gas sensitive layer of the ammonia gas sensor based on the small-period long-period fiber grating responds to the change of the ammonia concentration, and ammonia concentration information is displayed by the spectrometer.

6. An ambient temperature test system of the ammonia gas sensor according to claim 1, comprising a broadband light source, an optical circulator, the ammonia gas sensor based on the small-period long-period fiber grating, a spectrometer, and a tube furnace; wherein the broadband light source, the ammonia gas sensor based on the small-period long-period fiber grating, the spectrometer, and the optical circulator are arranged and connected according to positions, the ammonia gas sensor based on the small-period long-period fiber grating is placed in the tube furnace, an emitted light of the broadband light source is transmitted to the ammonia gas sensor based on the small-period long-period fiber grating through the optical circulator, a wavelength of a high-order Bragg resonance peak in a reflection spectrum of the ammonia gas sensor based on the small-period long-period fiber grating changes with a temperature for a temperature detection, and temperature information is displayed by the spectrometer.

7. The preparation method according to claim 4, wherein the high molecular polymer of the gas sensitive layer is poly(diallyldimethylammonium chloride) and the poly(acrylic acid).

8. The preparation method according to claim 4, wherein in the ammonia gas sensor, the small-period long-period fiber grating is obtained by inscribing at the interior of the single-mode fiber without a coating layer through a femtosecond laser adopting a line-by-line method, a transmission spectrum of the small-period long-period fiber grating contains high-order cladding modes, and a reflection spectrum of the small-period long-period fiber grating contains a series of high-order Bragg reflection peaks.

9. The ammonia concentration test system according to claim 5, wherein in the ammonia gas sensor, the high molecular polymer of the gas sensitive layer is poly(diallyldimethylammonium chloride) and poly(acrylic acid).

10. The ammonia concentration test system according to claim 5, wherein in the ammonia gas sensor, the small-period long-period fiber grating is obtained by inscribing at the interior of the single-mode fiber without a coating layer through a femtosecond laser adopting a line-by-line method, a transmission spectrum of the small-period long-period fiber grating contains high-order cladding modes, and a reflection spectrum of the small-period long-period fiber grating contains a series of high-order Bragg reflection peaks.

11. The ambient temperature test system according to claim 6, wherein in the ammonia gas sensor, the high molecular polymer of the gas sensitive layer is poly(diallyldimethylammonium chloride) and poly(acrylic acid).

12. The ambient temperature test system according to claim 6, wherein in the ammonia gas sensor, the small-period long-period fiber grating is obtained by inscribing at the interior of the single-mode fiber without a coating layer through a femtosecond laser adopting a line-by-line method, a transmission spectrum of the small-period long-period fiber grating contains high-order cladding modes, and a reflection spectrum of the small-period long-period fiber grating contains a series of high-order Bragg reflection peaks.
